(19) United States
(12) Patent Application Publication
(10) Pub. No.: US 2026/0137897 A1
(43) Pub. Date: May 21, 2026

(54) SYSTEM AND METHOD FOR DELIVERING COGNITIVE BEHAVIOUR THERAPY FOR INSOMNIA

(71) Applicant: RESMED ASIA PTE. LTD., Singapore (SG)

(72) Inventors: Faizan Javed, Bella Vista (AU); Bao Hui Lee, Singapore (SG); Jai Clayton Carey, Bella Vista (AU); Matthew John Backler, Mount Maunganui (NZ)

(21) Appl. No.: 19/390,304

(22) Filed: Nov. 14, 2025

(30) Foreign Application Priority Data
Nov. 15, 2024 (AU) 2024903765

(51) Int. Cl. A61M 21/02; A61M 21/00; G16H 20/70; G16H 40/67

(52) U.S. Cl. CPC A61M 21/02; G16H 20/70; G16H 40/67; A61M 2021/0083; A61M 2205/3303; A61M 2205/3306; A61M 2205/3584; A61M 2205/502

(57) ABSTRACT

The present disclosure is concerned with a system for delivering CBT-I to a patient. The system comprises one or more wearable devices each having one or more sensors configured to continuously monitor the patient's physiological, behavioural and/or environmental data; a patient application executable on a patient system, the patient application including one or more CBT-I modules and being configured to receive physiological, behavioural and/or environmental data from the wearable devices; a clinician application executable on a clinician system and configured to receive one or more CBT-I parameters pertinent to the CBT-I; and a CBT-I server communicatively coupled to the patient application and to the clinician application, the CBT-I server being configured to make physiological data received from the patient application available to the clinician application and selectively deliver one or more of the CBT-modules on the patient application in accordance with the CBT-I parameters received from the clinician application.

### CROSS REFERENCE TO RELATED APPLICATIONS

The present application claims the benefit of priority under 35 U.S.C. § 111 to Australian Patent Application No. 2024903765, filed on Nov. 15, 2024, the entire contents of which are incorporated herein by reference in their entirety.

### TECHNICAL FIELD

The present disclosure relates generally to systems and methods for treating insomnia and potentially other sleep disorders. The present disclosure relates more specifically, but not exclusively, to systems and methods for treating insomnia through cognitive behavior therapy for insomnia.

### BACKGROUND

Insomnia is a prevalent sleep disorder that can profoundly impact the mental and physical health of sufferers. It is estimated that 21.5% of the adult population of the United States suffers from insomnia. Characterized by difficulty falling asleep, staying asleep, or waking up too early, insomnia is implicated in sufferers experiencing feelings of exhaustion, reduced cognitive function, irritability and a general decline in quality of life. While lifestyle changes and medications can temporarily relieve some insomnia related symptoms, a highly effective long-term treatment for chronic insomnia is Cognitive Behaviour for Insomnia (CBT-I). This structured, evidence-based therapeutic approach targets the root psychological and behavioral causes of insomnia, offering patients a sustainable pathway to restful sleep without the dependency issues associated with pharmacological treatment modalities.

Insomnia can be either acute, lasting a few days to weeks due to stress or other situational factors, or chronic, persisting for three or more nights per week over a period of three months or longer. Chronic insomnia is of particular concern due to its cumulative impact on health. Studies link it to various physical conditions, including hypertension, obesity, and immune system dysfunction, as well as mental health disorders such as anxiety and depression. People suffering from chronic insomnia often experience a diminished capacity for memory, focus, and decision-making. The wide-ranging consequences of insomnia on both individual and societal levels underscore the need for effective treatment approaches.

Historically, treatments for insomnia have included lifestyle modifications and sleep hygiene practices, such as establishing a regular sleep schedule, reducing caffeine intake, and creating a comfortable sleep environment. For more severe cases, sedative-hypnotic medications are sometimes prescribed. However, while these medications can provide short-term relief they can carry a high risk of dependency, tolerance and potential withdrawal symptoms. Additionally, pharmacological therapies do not address underlying behavioral and cognitive factors that can contribute to insomnia. In view of these significant limitations sleep specialists are increasingly advocating for non-pharmacological therapies and in particular CBT-I.

CBT-I is specifically designed to treat insomnia and is a structured therapy that targets the maladaptive thoughts and behaviours that perpetuate poor sleep. Rather than masking symptoms, CBT-I assists patients in re-establishing healthy sleep patterns and addresses underlying psychological barriers.

Despite its benefits, there are improvements that can be made to CBT-I so that it is more personalized to the patient and responsive to their individual insomnia symptoms.

### SUMMARY

In one aspect, the present disclosure is concerned with a system for delivering CBT-I to a patient, comprising: one or more wearable devices each having one or more sensors configured to continuously monitor the patient's physiological, behavioural and/or environmental data; a patient application (app) executable on a patient system, the patient app including one or more CBT-I modules and being configured to receive physiological, behavioural and/or environmental data from the one or more wearable devices; a clinician application executable on clinician system, the clinician application being configured to receive one or more CBT-I parameters pertinent to the CBT-I; and a CBT-I server communicatively coupled to the patient app and to the clinician application, the CBT-I server being configured to make physiological data received from the patient app available to the clinician application and selectively deliver one or more of the CBT-modules on the patient app in accordance with one or more CBT-I parameters received from the clinician application.

In some embodiments, the CBT-I server includes an analytics module configured to process physiological data received from the patient app and CBT-I parameters received from the clinician application and generate a CBT-I regime that selectively delivers one or more of the CBT-I modules on the patient app.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a block diagram illustrating a networked computing environment in which aspects of the present invention may be implemented;

FIG. 2 is a schematic illustration of a wearable device in accordance with an embodiment of the present invention and exemplary sensors that the wearable device incorporates;

FIG. 3 is a schematic illustration of the wearable device sensors configured to continuously monitor a patient's physiological and behavioural processes over a 24 hour period;

FIG. 4 is a schematic illustration of a user interface for visualizing sensor data in a Clinician Application in accordance with an embodiment of the present invention;

FIG. 5A-5H illustrates a use of the present invention to deliver CBT-I to perform circadian alignment;

FIG. 6A-6H illustrates a use of the present invention to deliver CBT-I to perform sleep compression;

FIG. 7A-7H illustrates a use of the present invention to deliver augmented CBT-I to detect and respond to a hyperarousal event.

FIG. 8A-8H illustrates a use of the present invention to facilitate light exposure as a component of a CBT-I regime;

FIG. 9 is a block diagram illustrating a computer system, which may be used to implement various embodiments of the present invention.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

In the following description, for the purposes of explanation, numerous specific details are set forth to provide a thorough understanding of the present invention. It will be apparent, however, that the present invention may be practiced without these specific details. In some instances, well-known structures and devices are shown in block diagram form to avoid unnecessary obscuring.

Embodiments of the present invention provide a system for delivering CBT-I to a patient. In broad terms, the system comprises one or more wearable devices, a patient application (app) and a clinician application both communicatively coupled to a CBT-I server application executing on a CBT-I server.

The wearable device is worn by the patient and comprises one or more sensors for measuring physiological data and behavioural data from the patient, as well as environmental data. The measured physiological, behavioral and/or environmental data are relevant to the CBT-I that the patient is undergoing in terms of both delivering particular CBT-I components and measuring their effectiveness. The wearable device also includes one or more sensory actuators for providing sensory actuation or feedback (such as haptic, visual, auditory or other feedback) to the patient as an aspect of the CBT-I therapy. Sensory actuation or feedback is also used to trigger a prompt to the patient to enter a cognitive input into the patient app relevant to the insomnia-related event to which the sensory activation relates.

The patient app is used to control the wearable device, receive data from the sensors and transmit the data to the CBT-I server application, transmit sensory actuation commands to the wearable device, as well as serving as an application for delivering the various components of a CBT-I program to the patient.

The clinician application allows the clinician to deliver a personalized CBT-I program to each patient and access the physiological data and patient cognitive inputs to selectively deliver CBT-I components to the patient and evaluate their effectiveness. The clinician application includes modules for the clinician to input fundamentals in terms of the CBT-I treatment. The clinician application also analyses each individual patient's data and recommends CBT-I components to the clinician that are personalized to the patient. The clinician may then choose to deliver the recommended CBT-I components to the patient modified as needed in accordance with clinical decisions. The clinician application thus functions as a clinician-decision support tool which reduces clinician time/energy needed to analyse data and make appropriate CBT-I delivery decisions.

Both the patient app and clinician application are communicatively coupled to the CBT-I server application. The CBT-I server application continuously receives the physiological and behaviour data collected from the sensors of the wearable device. The CBT-I server application maintains records of each patient undergoing CBT-I treatment and stores the received data in association with each patient's record. The CBT-I server application also has access to an analytics module (which typically includes one or more machine learning models) that process the data and select appropriate CBT-I components to cause the patient to apply behavioural adjustments. The processed data also allows the clinician to evaluate the effectiveness of the CBT-I program.

In this regard, CBT-I is built on several core components, namely sleep education, sleep restriction, intensive sleep retraining, stimulus control, cognitive restructuring, relaxation techniques and lifestyle influences.

Sleep education involves the clinician educating patients about the biology of sleep, including the sleep-wake cycle and the importance of maintaining consistent sleep patterns. Understanding the natural cycle of sleep can alleviate the patient's anxieties and misconceptions, providing a foundation for the behavioural adjustments that follow.

Sleep restriction is responsive to a clinical observation that those suffering from insomnia spend extended periods in bed in the hope of increasing sleep. The strategy often backfires however, by weakening the brain's association between bed and sleep. Sleep restriction involves limiting time in bed to match actual sleep time, thereby building stronger sleep pressure. Gradually, as sleep quality improves, the patient's time in bed is increased.

Intensive sleep retraining (ISR) was developed based on stimulus control therapy, which aims to reverse anxiety around sleep by associating the bed with rapid sleep onset. The traditional approach involves going to bed only when sleepy, getting out of bed if sleep doesn't come within 15-20 minutes, and waking at a consistent time each morning, even with limited sleep. ISR condenses these sleep trials into a single night, giving 30-40 sleep onset experiences in one night, compared to ˜20 experiences over two weeks with standard stimulus control therapy. Combining ISR with stimulus control has proven to be even more effective.

Stimulus control aims to re-establish a strong mental connection between the bed and sleep. Patients are instructed to use the bed only for sleep and to avoid activities such as watching television, reading or worrying in bed. Additionally, if a patient cannot fall asleep after a prescribed time (for example 20 minutes), they are encouraged to leave the bed and engage in calming activity until they feel sleepy. This technique reduces the anxiety and frustration that often accompanies prolonged wakefulness in bed.

Cognitive restructuring seeks to address the fact that anxiety about sleep itself can be a significant barrier to restful nights. Cognitive restructuring involves identifying and challenging negative thoughts and beliefs about sleep, such as catastrophic thinking (such as the thought that not sleeping well will inevitably ruin the next day). By reframing these thoughts in a more realistic, positive way, patients can reduce pre-sleep anxiety and develop a more positive outlook on their sleep abilities.

Relaxation techniques performed pre-sleep are integral to easing the transition to restful sleep. Clinicians teach relaxation techniques such as deep breathing exercises, progressive muscle relaxation and mindfulness meditation, which can calm both mind and body, reducing the arousal that contributes to insomnia. In the present disclosure, relaxation techniques can be taught and delivered through the patient app.

CBT-I regimes that are delivered to the patient through the patient app also seek to reduce physiological stress inputs that can impact the body's sleep drive and the patient's readiness for bed.

CBT-I also utilizes a sleep diary to track sleep patterns, identify sleep-related issues and measure progress over time. The sleep diary is maintained through the patient app. By recording details about their sleep (typically each morning), the patient and the clinician gain a clearer understanding of sleep habits and patterns that may contribute to insomnia.

At commencement of CBT-I, patients typically complete a sleep diary for one to two weeks to establish a baseline. This initial record reveals patterns in sleep and wake times, total sleep duration, nighttime awakenings, and factors influencing sleep quality. The analytics module in the CBT-I server application analyses the sleep diary to pinpoint specific issues, such as inconsistent sleep schedules, prolonged awakenings during the night, or spending too much time in bed awake. This assists in tailoring CBT-I interventions to the individual patient.

The sleep diary also typically tracks the time spent asleep versus time in bed, allowing the clinician to compute sleep efficiency. Monitoring sleep efficiency over time assists the clinician to evaluate the effectiveness of CBT-I components such as sleep restriction, in the course of gradually increasing time in bed as sleep efficiency improves. Completing a sleep diary also assists the patient to take an active role in therapy. In particular, the sleep diary encourages the patient to be mindful of their sleep habits and to adhere to clinician-recommended behavioural changes such as consistent wake times or limiting time in bed.

Throughout the course of CBT-I, the sleep diary serves as a record of progress, assisting both the patient and clinician to see improvements, such as shorter time to fall asleep, fewer awakenings, and/or increased sleep efficiency.

A typical sleep diary includes entries for bedtime, estimated time to fall asleep, wake time, number of awakenings and perceived sleep quality. This systematic approach makes the sleep diary a valuable component of CBT-I, offering insights that help drive effective, personalized treatment for insomnia.

The present disclosure augments the typical CBT-I program by including a biofeedback collection component. The component continuously collects physiological and behavioural data from sensors in a wearable device over 24 hour periods. This enables the system to measure the impact of events occurring at different times during the day on sleep performance. In this regard, how a person responds to events occurring, for example during the day, can be implicated with insomnia symptoms. Monitoring over 24 hour periods allows the clinician to build up a picture of behaviours and interactions that with ameliorating incidents of insomnia.

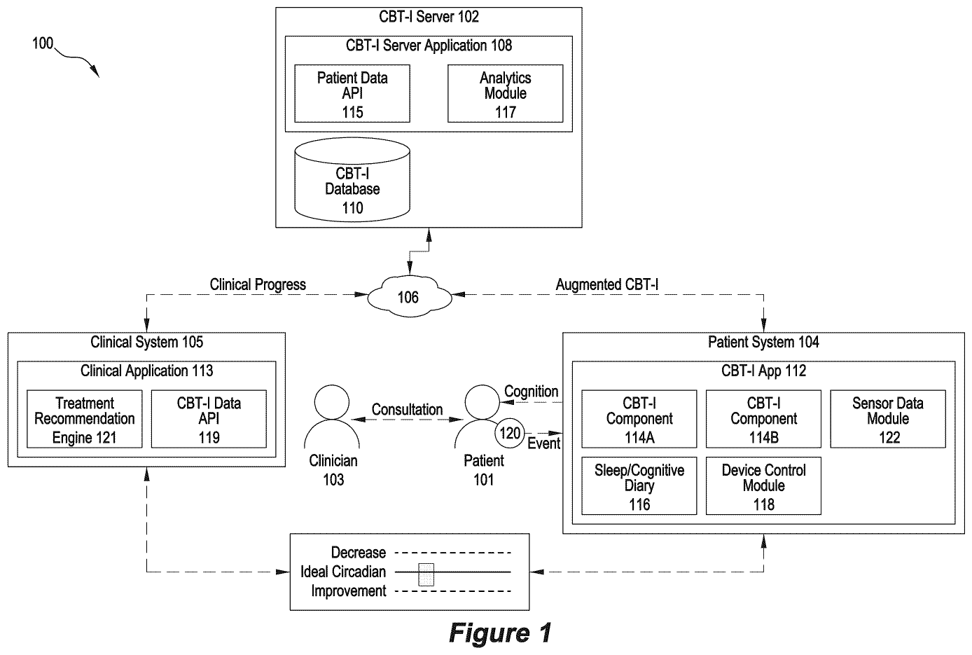

FIG. 1 illustrates a computing environment 100 in which aspects of the present disclosure are implemented. The environment 100 is a networked environment comprising a CBT-I Server 102 in communication with a Patient System 104 and a Clinician System 105 over one or more communication networks 106. Aspects of the computer processing described below are performed by a CBT-I Server Application 108 executing on the CBT-I Server 102, a CBT-I App 112 executing on the Patient System 104 and a Clinician Application 113 executing on the Clinician System 105.

CBT-I Server 102 further includes a CBT-I Database 110 on which patient records for the clinician's patient 101 undergoing CBT-I are stored. Each patient's record also includes current and archived physiological and behavioural data collected by sensors. CBT-I Database 110 is typically a storage medium such as a hard drive (or collection of hard drives). A database management system (not shown) executing on CBT-I Server 102 implements a database on CBT-I Database 110 for storing and retrieving data managed by the CBT-I Server Application 108.

CBT-I Server 102 makes available a Patient Data API endpoint 113 that the Patient System 104 and Clinician System 105 use to upload data (typically that collected by sensors) to the CBT-I Server Application 108.

CBT-I Server 102 further includes an Analytics Module 117. Analytics Module 117 incorporates machine learning models, algorithm libraries, expert systems, lookup tables and combinations thereof to analyse physiological, behavioural and other data that is uploaded to the CBT-I Server 102. The Analytics Module 117 utilises the analysed data in a variety of applications including to generate personalized CBT-I interventions for the patient 101. The personalized CBT-I interventions take the form of evidence-based treatment recommendations that the Analytics Module 117 communicates to the Clinician Application 113 at a CBT-I Data API endpoint and/or (more often) to the CBT-I app 112. The Clinician Application 113 also includes a Treatment Recommendation Engine 121 that generates CBT-I interventions for the patient using the data received at the CBT-I API endpoint 119 and serves an input for CBT-I treatment parameters that the clinician 103 enters.

The Analytics Module 117 and/or Treatment Recommendation Engine 121 are also configured to synthesize patient data for rapid review and interpretation by the clinician 103 through the Clinician Application 113. The clinician 103 is able to review the analyzed data and monitor the patient's progress in the delivered CBT-I both during a consultation and outside of the consultation, for example to periodically review and monitor the patient's 101 progress in the CBT-I therapy.

CBT-I Server 102 has been illustrated as a single system. CBT-I Server 102 can, however, be a scalable server system comprising multiple nodes which can be commissioned/decommissioned based on processing demands. Typically, server systems are server computers that provide greater resources (e.g. processing, memory, network bandwidth) in comparison to client systems such as the Patient System 104 and Clinician System 105.

In the illustrated embodiment, CBT-I Database 110 is illustrated as part of the CBT-I Server 102. However CBT-I Database 110 could be a separate system in operative networked communication with the CBT-I Server 102. For example, the CBT-I Database 110 could be a networked-attached storage device, an entirely separate storage system accessed via a database management system, or any other appropriate data storage mechanism.

As described in further detail below, the CBT-Server Application 108 performs various operations in response to commands received from (and initiated at) the CBT-I App 112 and Clinician Application 113. As such, when executed by the CBT-I Server 102, the CBT-I Server Application 108 configures the CBT-I Server 102 to provide server-side functionality to the CBT-I App 112 and Clinician Application 113. To provide this functionality, the CBT-I Server Application 108 comprises one or more suitable application programs, libraries, or other software infrastructure.

Where the CBT-I App 112 and Clinician Application 113 are applications that run in web browsers, CBT-I Server Application 108 will typically be, or interact with, a web server such as a server implemented with the node. js runtime environment. Where the client CBT-I App 112 and Clinician Application 113 are dedicated applications provided specifically to interact with the CBT-I Server Application 108 (for example when the CBT-I App 112 is an app that runs on a mobile device and the Clinician Application 113 is an application that runs on a desktop computer), the CBT-I Server Application 108 will typically be, or interact with, an application server. CBT-I Server 102 may be provided with both web server and application server applications to enable it to serve both web browser and dedicated client applications.

The CBT-I Server 102, Patient System 104 and Clinician System 105 communicate data between each other either directly or indirectly through one or more Communications Networks 106. Communications Network 106 may comprise a local area network (LAN), a public network (such as the Internet), or a combination of networks.

While only one Patient System 104 and Clinician System 105 are depicted in Environment 100, a typical environment would typically include many more Patient Systems 104 and additional Clinician Systems 105 collectively served by the CBT-I Server 102. Typically, multiple patients (each with their own Patient System 104) are served by a single Clinician System 105 that is operated by a single clinician or installed at a clinic and operated by multiple clinicians. The Clinician System 105 can also be integrated into other medical management software such as software platforms that are used to manage sleep labs and clinics.

While the Patient System 104 can be any type of computer system, including a desktop computer or laptop computer, it will more commonly be a smartphone or a tablet device. The Clinician System 105 is more commonly a desktop computer or laptop computer.

When executed by the Patient System 104, the CBT-I App 112 configures the Patient System 104 to provide client-side CBT-I functionality and interact with the CBT-I Server 102 (or, more specifically, the CBT-I Application 108 executing thereon).

The CBT-I App 112 and Clinician Application 113 may be general web browser applications (such as Chrome, Edge, Safari or the like) which access the CBT-I Server Application 108 via an appropriate uniform resource locator (URL) and communicate with the CBT-Server Application 108 via general world-wide-web protocols (e.g. http, https, ftp) and application programming interfaces (APIs) (e.g. REST APIs). Alternatively, the CBT-I App 112 and Clinician Application 113 may be specific apps/applications programmed to communicate with the CBT-Server Application 108 using defined API calls.

A given Patient System 104 and Clinician System 105 may each have more than one client application 112 and 113 respectively installed thereon, for example both a general web browser application and a dedicated programmatic client application.

The CBT-I App 112 also includes modules to deliver the CBT-I treatment to the patient 101. In this regard, the CBT-I App 112 includes a plurality of CBT-I Components that each deliver a component of the CBT-I treatment to the patient 101. Two CBT-I Components 114A and 114B are shown for the purpose of illustration. Those skilled in the art will appreciate that the CBT-I App 112 invariably includes a range of additional CBT-I Components (for example components for sleep application, sleep restriction, stimulus control, cognitive restructuring, light exposure management, relaxation techniques, goal setting, sleep coaching, sleep schedules and gamified components such as for delivering gamified cognitive brain training exercises).

The CBT-I App 112 also includes a Sleep/Cognitive Diary Module 116 for the patient 101 to maintain an electronic sleep diary containing the sleep and insomnia-related data points discussed above and an electronic cognitive diary to record and acknowledge daytime influences. The patient 101 enters daytime influences and acknowledgements thereof into the Sleep/Cognitive Diary 116 either of their own accord or in response to prompts from the CBT-I App 112 or from a connected wearable device. Information that the patient 101 enters into the Sleep/Cognitive Diary 116 is made available to the Analytics Module 117 for processing to thus contribute to the patient's personalized CBT-I treatment regime.

The CBT-I App 112 further includes a Device Control Module 118 that is paired with a wearable device 120 that the patient 101 wears during the course of the CBT-I treatment regime. The wearable device 120 is equipped with a plurality of sensors (discussed in further detail below) that continuously monitor the patient's 101 physiological functions with a particular emphasis on monitoring circadian health biomarkers. The wearable device 120 transmits sensor data captured by the sensors to a Sensor Data Module 122 within the CBT-I App 112. The CBT-I App then suitably uploads the sensor data to the CBT-I Server Application 108 for processing by the Analytics Module 117 and conversion into real-time actionable insights. As illustrated in FIG. 1, there is a circular, three-way conversation between the patient 101, clinician 103, and the sensor data, involving the sensors of the wearable device 120 measuring sensor data and detecting an event, the wearable device communicating the event to the Analytics Module 117 by way of the CBT-I App 112, the Analytics Module 117 processing the sensor data and generating an augmented CBT-I strategy for the patient that typically involves activating and/or suppressing one or more of the CBT-I Components and the clinician 103 monitoring the effectiveness of the strategy through the Clinician Application 113 during the consultation.

The wearable device 120 can take a variety of forms including being worn like a necklace around the patient's 101 neck and contacting the patient's upper chest. The wearable device 120 can also be attached to the patient 101 by way of a patch that contacts the patient's skin at a suitable location such as the upper arm, wrist or the back of the neck.

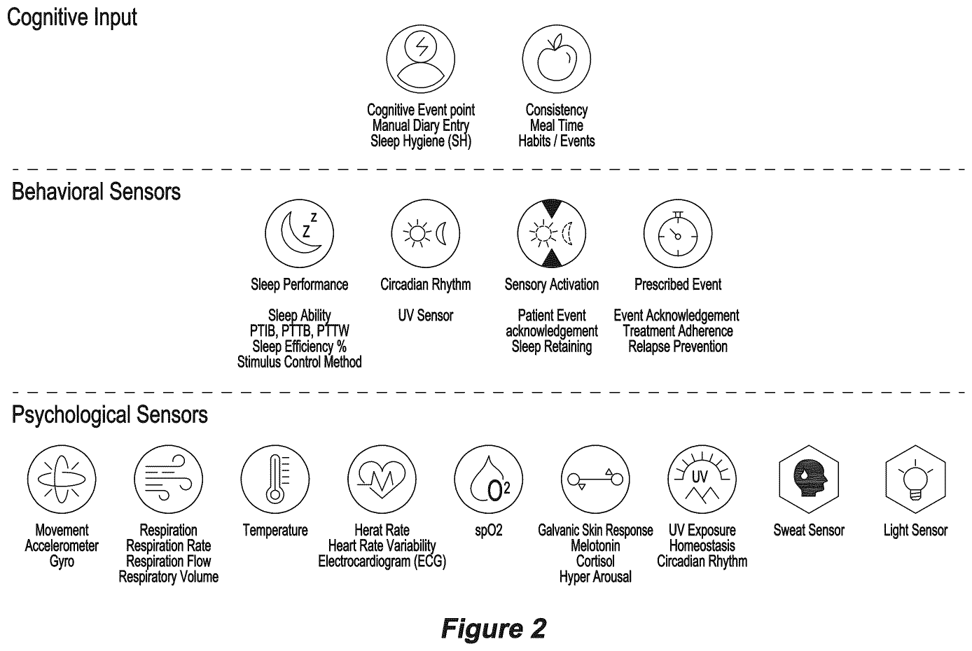

The various physiological sensors that are incorporated in the wearable device 120 are illustrated by reference to FIG. 2. FIG. 2 illustrates:
A movement sensor such as an accelerometer or gyroscope for detecting movement.
A respiration sensor for measuring respiratory rate, respiratory flow, respiratory volume, inspiration and expiration.
A temperature sensor for measuring temperature, including skin and/or core body temperature.
A heart rate sensor (for example an electrocardiogram) for measuring heart rate and heart rate variability.
An SPO2 sensor (ie. pulse oximeter for measuring the oxygen saturation level in the blood.
A galvanic skin response sensor for measuring changes in the electrical conductance of the skin which vary with moisture levels. The response is primarily influenced by sweat gland activity which is controlled by the sympathetic nervous system and often increases with emotional arousal or stress. As such, measuring galvanic skin response can provide insights into phenomena (such as hyper arousal and stress) that are implicated with insomnia symptoms. The galvanic skin response also provides a useful biofeedback signal to the Analytics Module 117 to generate an augmented CBT-I strategy for the patient 101.
A light exposure sensor for measuring the quantity of different wavelengths of light contacting the patient. Light exposure is directly related to exposure to sunlight which is a major signal for the body's internal clock, assisting with regulating the circadian rhythm. Sunlight exposure during the day, particularly in the morning assists with suppressing melatonin production, signaling wakefulness and alertness. As daylight fades, melatonin levels rise, helping prepare the body for sleep. As such, measuring various lights to which the patient is exposed is useful for providing insights into the patient's sleep-wake cycle and other circadian processes.
A sweat sensor for capturing sweat data from the skin and detecting and quantifying hormone levels, principally melatonin and cortisol. Melatonin and cortisol are each useful biomarkers for monitoring and treating insomnia. In this regard, monitoring cortisol and/or melatonin levels provides insights into the time of occurrence of the patient's sleep-wake cycle. Measuring melatonin levels can also assist the Analytics Module 117 to determine that the patient's insomnia is related to a melatonin deficiency. The sweat sensor also detects and quantifies other substances such as caffeine.
A light sensor for measuring the patient's exposure to visible light including from artificial sources.

Components of the computing environment 100 also incorporate a number of “behavioural sensors” and actuators, namely:
Sleep Performance Input and Computation refers to the functionality of the Clinician Application 113, through which the clinician enters CBT-I parameters to be actioned in the CBT-I treatment. Inputs include sleep ability, prescribed time in bed, prescribed time to bed, prescribed time to wake, sleep efficiency and stimulus control methods. Sleep efficiency is a metric used to assess the quality of sleep by comparing the amount of time a person spends sleeping to the total time they spend in bed. It is expressed as a percentage and serves as an indicator of how effectively a person is using their time in bed for actual sleep. High sleep efficiency generally indicates restful, uninterrupted sleep, while low sleep efficiency may suggest issues with falling asleep, staying awake, or waking up too early.
Circadian rhythm, through which insights into the patient's circadian rhythm are derived through gathering data such as from the UV, light, sweat, saliva, movement and heartrate sensors and performing analytics thereon.
Sensory Activation, relating to the sensory actuators in the wearable device 120 that initiate sensory actuation to the patient as an aspect of the CBT-I therapy such as to indicate the occurrence of an event detected by the physiological sensors. The patient 101 typically acknowledges the occurrence of the event on the wearable device 120 or in the CBI-I App 112. The CBT-I App 112 may also prompt the patient 101 to enter a cognitive input related to the event into the Sleep/Cognitive Diary 116.
Prescribed Event, relating to event acknowledgment, treatment adherence and relapse prevention.

The patient typically enters additional cognitive inputs into the Sleep/Cognitive Diary including manual diary entries, sleep hygiene information and consistency indications related to life activities such as the time meals are taken and other habits and events.

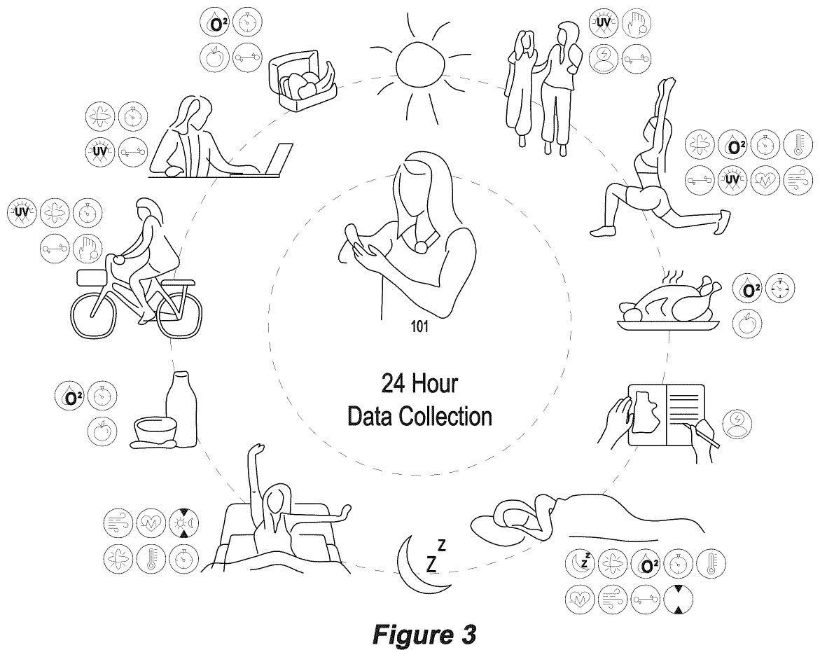

As illustrated in FIG. 3, the sensors in the wearable device are configured to continuously monitor the patient's 101 physiological and behavioural processes over a 24 hour period. However, individual sensors may become more or less active at different times during the 24 hour period. In this regard, as illustrated in FIG. 3, more of the physiological sensors may actively monitor the patient's at the time of going to sleep and during sleep compared to when the patient is eating a meal or making a cognitive input in the Sleep Diary.

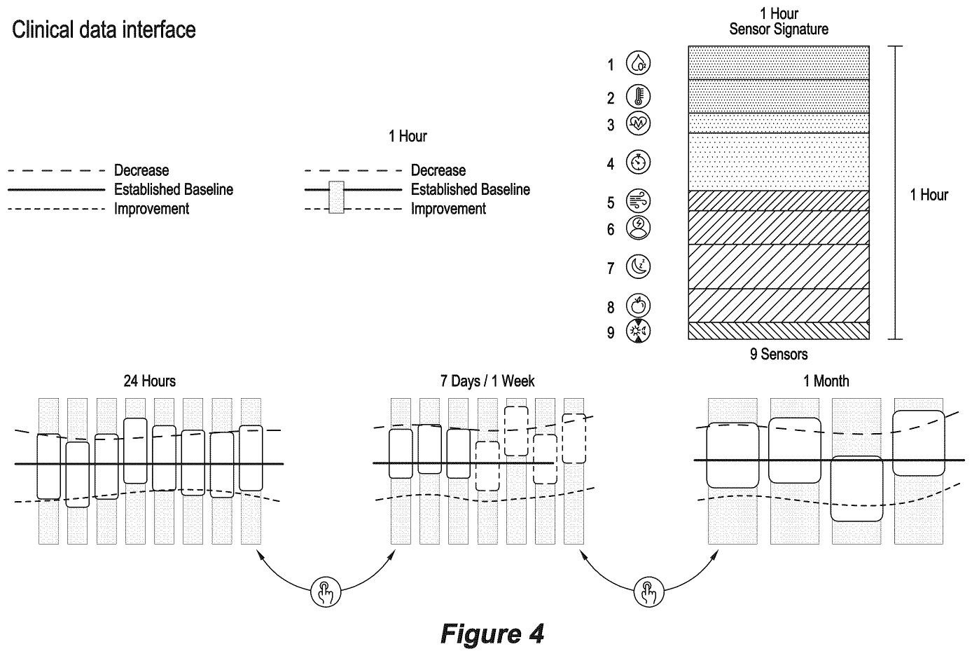

FIG. 4 shows an embodiment of a user interface for visualizing the sensor data in the Clinician Application 113 and/or CBT-I App 112. The clinician 103 typically delivers CBT-I by having the patient 101 wear the wearable device 120 for a period of time to gather an initial set of sensor data to establish an initial baseline for the patient's 101 biomarkers, circadian rhythm, sleep behaviours and cognitive inputs. Once the baseline is established, the Clinician Application 113 is placed into a monitoring mode, in which the sensors of the wearable device 120 continuously monitor the patient's physiological functions with a particular focus on circadian-rhythm related functions.

The Clinician Application 113 divides the 24 hour monitoring cycle into hourly analysis units characterized by the relative contribution of individual sensors to the monitoring during the analysis unit. The illustrated embodiment shows an example analysis unit where 9 sensors were active with the areas of the different rectangles showing each sensor's relative contribution to the monitoring. Overlaying adjacent analysis units over the patient's 101 baseline allows the clinician to analyse when and to what extent the sensors detected deviations (in terms of decreases and improvements) from the established baseline. Visualizing sensor data in this way also allows the clinician to inspect how the data changes over the course of 24 hours as the patient 101 undertakes different activities. It also allows the clinician to identify anomalies in the data, such as where the measured physiological data does not coincide with the patient's 101 expected activities for example at a particular time of the day.

The Clinician Application 113 also divides the monitoring activity into analysis units of different duration. As shown in FIG. 4, analysis units of 1 day duration can be created and adjacent units displayed on the Clinician System 105. Analysis units of 24 hour duration displayed over a one week period allows the clinician to identify longer term trends and utilize the data to inform diagnostic or evaluative decisions.

Analysis units of 7 days duration displayed over a one month time period are also possible.

In each case, the user interface can display when a particular CBT-I Component was activated (shown in green in FIG. 4) and the impact of the Component on the measured data.

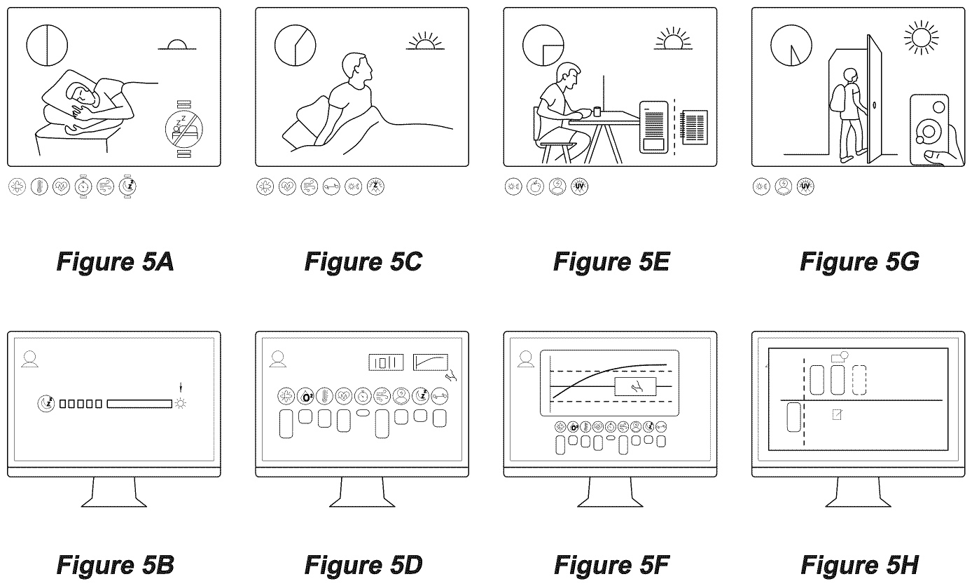

FIG. 5 illustrates a use of the present disclosure to deliver CBT-I to perform circadian alignment.

In FIG. 5A, the patient 101 is wearing the wearable device 120 at sunrise with six sensors active. Circadian adjustment is activated at which time the wearable device 120 provides sensory activation to awake the patient. Sunrise time and prescribed time to wake were previously entered into the Clinician Application 113 to coordinate the sensory application. FIG. 5B shows the user interface displayed on the Clinician System 105 at the time of sensory activation. The Clinician Application 113 also records user alignment to the sun and health circadian.

In FIG. 5C, the patient 101 is shown waking and beginning their day. The patient 101 wakes and responds to the sensory activation in the wearable device 120 and/or CBT-I App 112. Cumulative UV exposure measurement begins. FIG. 5D shows the user interface displayed on the Clinician System 105 depicting all active sensor data being recorded as well as the patient's sleep and waking patterns.

FIG. 5E shows the patient 101 logging a diary entry in the Sleep/Cognitive Diary 116 about their night's sleep, breakfast and how they are feeling. FIG. 5F shows the user interface displayed on the Clinician System 105 depicting the importance of circadian re-alignment in helping enhance the users night time sleep outcomes.

FIG. 5G shows a successful circadian alignment which is indicated by a healthy start to the day, allowing the patient to begin their day with confidence. FIG. 5H shows the user interface displayed on the Clinician System 105 depicting a full 24 hour circadian rhythm with cumulative light (including UV) exposure for the clinician 103 to assess and encourage progress.

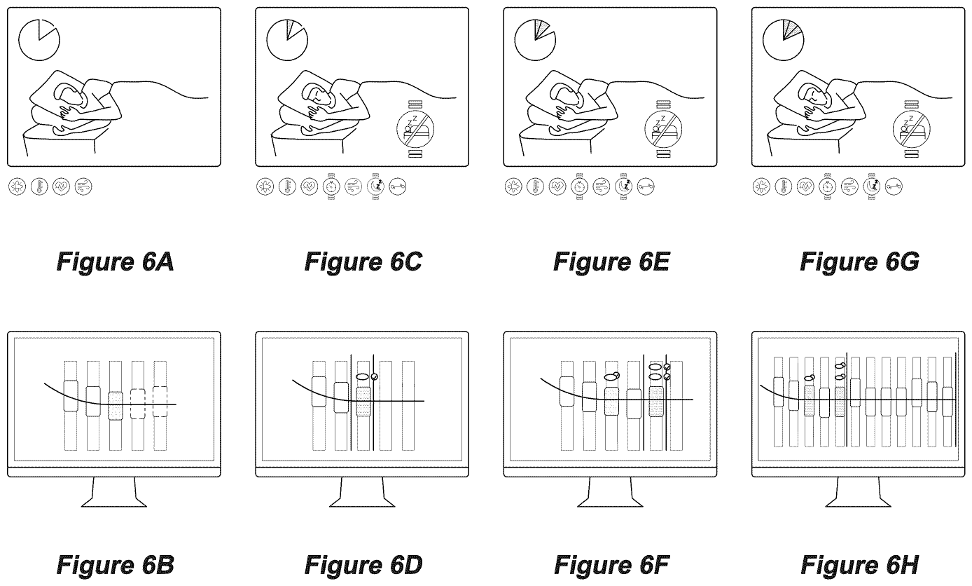

FIG. 6 illustrates a use of the present disclosure to deliver CBT-I to perform sleep compression.

In FIG. 6A, the patient 101 is wearing the wearable device 120 and commences intensive sleep retraining. Following the baseline, the patient 101 starts the wearable device 120 for intensive retraining. FIG. 6B shows the user interface displayed on the Clinician System 105 when the wearable device commences intensive sleep retraining.

In FIG. 6C, the patient 101 receives a sleep restriction prompt from the wearable device 120. The wearable device 120 sets an optimum sleep period from the established baseline and prompts the user to stay awake. FIG. 6D shows the user interface displayed on the Clinician System 105 depicting the wearable device detecting a sleep physiology event and detecting the patient 101 moving. Responses by the patient are recorded in the Clinician System 105 signaling adherence.

FIG. 6E shows the patient 101 responding to sensory activation from the wearable device 120 and indicating to the wearable device 120 that they are awake. FIG. 6F shows the user interface displayed on the Clinician System 105 depicting the wearable device 120 detecting additional sleep physiology events and the patient 101 moving. Sensory responses are registered by the wearable device 120 for the remaining prescribed time frame.

FIG. 6G shows sleep retraining continuing and the wearable device 120 continuing to support the patient 101 through retraining as the onset of sleep intensifies. FIG. 6H shows the user interface displayed on the Clinician System 105 depicting a full sleep retraining models and cognitive diary recording allowing for a successful clinician and patient intervention.

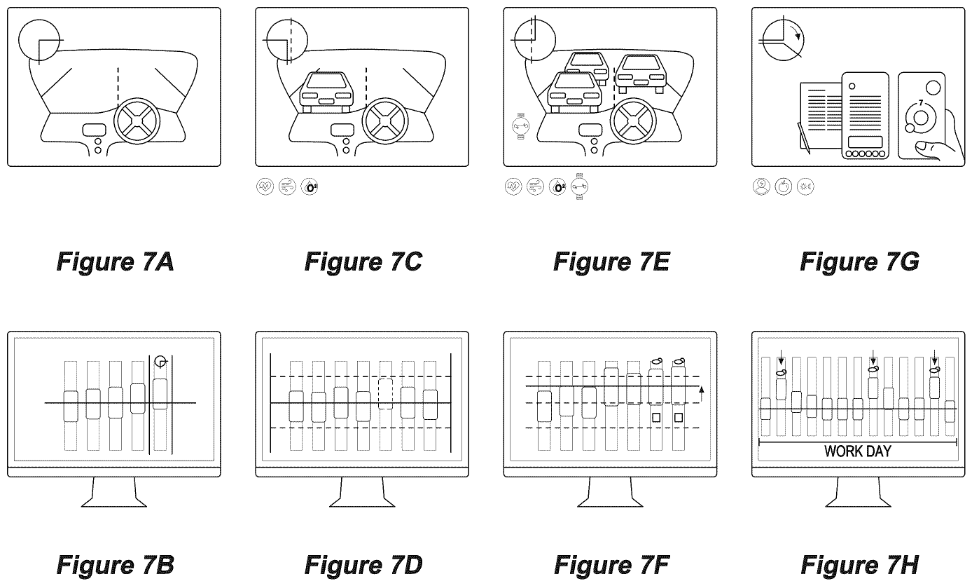

FIG. 7 illustrates a use of the present disclosure to deliver augmented CBT-I to detect and respond to a hyperarousal event.

In FIG. 7A, the patient 101 is wearing the wearable device 120 and performing a common daytime activity under elevated anxiety. In the illustrated embodiment, the activity is driving to work in traffic and becoming anxious about being late. FIG. 7B shows the user interface displayed on the Clinician System 105 when performing hyperarousal monitoring. In the illustrated embodiment, the wearable device 120 detects changes in physiological state relative to the baseline.

In FIG. 7C, the patient 101 experiences an anxiety-invoking condition that triggers the body to treat thoughts and emotions as threats. FIG. 7D shows the user interface displayed on the Clinician System 105 when a recording is made of physiological variations that cause elevated emotions that call for management by CBT-I Components.

FIG. 7E shows the wearable device 120 sensing the patient's 101 psychological state and intercepting with positive augmentation. For example, the CBT-I App 112 can activate a relevant CBT-I Component 114 (such as a relaxation exercise) that the patient 101 can perform to improve their psychological state and potentially improve sleep through psychological resolution of the event. FIG. 7F shows the user interface displayed on the Clinician System 105 when the Clinician System 105 records the patient inputting a cognitive user entry in response to the positive augmentation.

FIG. 7G shows the patient 101 entering a cognitive input in the Sleep/Cognitive Diary 116 recording the event and the cognitive reconciliation of it. FIG. 7H shows the user interface displayed on the Clinician System 105 providing advanced visibility for the clinician to augment CBT-I at the individual patient level. Continuously monitoring for hyperarousal events also allows the clinician to identify patterns in the occurrence of multiple events and prescribe suitable responsive CBT-I Components to prompt cognitive recognition and eventually behavioral change.

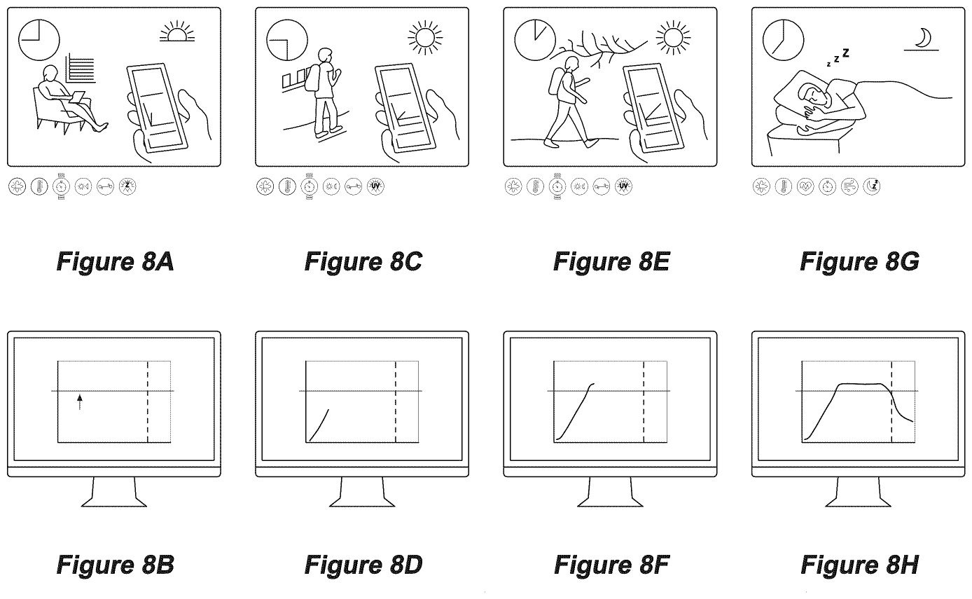

FIG. 8 illustrates a use of the present disclosure to facilitate light (including UV) exposure as a component of a CBT-I regime.

In FIG. 8A, the patient 101 is wearing the wearable device 120 and using the CBT-I App 112 which promotes early light (including UV) exposure as a strategy for early alignment to an ideal circadian rhythm. FIG. 8B shows the user interface displayed on the Clinician System 105 when circadian alignment is initiated. The Clinician System 105 sets a prescribed time to wake to align circadian and homeostatic sleep drive.

In FIG. 8C, the patient 101 enters a UV exposure goal in the CBT-I App 112 and the sensors in wearable device 120 measure light exposure and its relationship within CBT-I. FIG. 8D shows the user interface displayed on the Clinician System 105 as the sensors monitor light (including UV) exposure during the day and plot the exposure as it approaches the prescribed goal.

FIG. 8E shows the user interface displayed on the Patient System 104 prompting the patient to adjust their behaviour to meet the light exposure goal. The user interface also displays live graphical user goals to assist in promoting healthy UV behaviours. FIG. 8F shows the user interface displayed on the Clinician System 105 when the Clinician System 105 monitors cognitive behaviour and the prescribed time to bed parameter.

FIG. 8G shows the patient 101 developing an understanding and valuing the importance of UV exposure on sleep. FIG. 8H shows the user interface displayed on the Clinician System 105 demonstrating how 24 hour monitoring assists in increasing CBT-I adherence and positive sleep outcomes.

The present disclosure also includes functionality to detect the patient 101 becoming drowsy outside of prescribed sleep hours and initiate sensory activation to prevent the patient from falling asleep.

Furthermore, the present disclosure includes functionality to apply stimulus control and cognitive adaptation to the patient 101 as an aspect of a CBT-I regime.

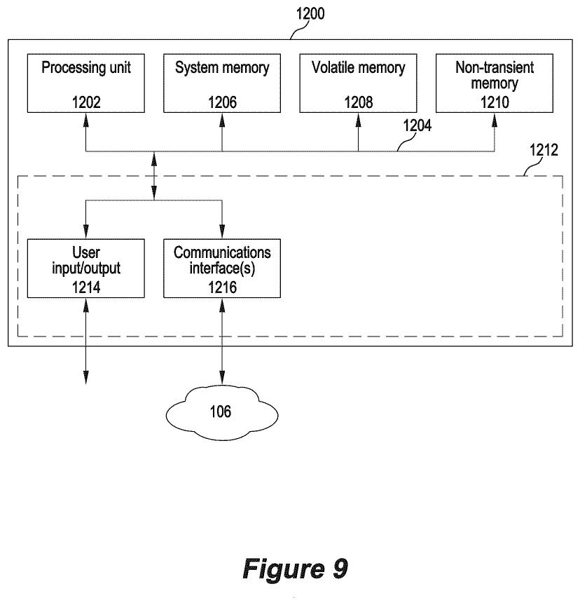

FIG. 9 provides a block diagram of a computer processing system 1200 configurable to implement embodiments and/or features described herein. System 1200 is a general purpose computer processing system. It will be appreciated that FIG. 9 does not illustrate all functional or physical components of a computer processing system. For example, no power supply or power supply interface has been depicted, however system 1200 will either carry a power supply or be configured for connection to a power supply (or both). It will also be appreciated that the particular type of computer processing system will determine the appropriate hardware and architecture, and alternative computer processing systems suitable for implementing features of the present disclosure may have alternative components to those depicted.

Computer processing system 1200 includes at least one processing unit 1202. The processing unit 1202 may be a single computer processing device (e.g. a central processing unit, graphics processing unit, or other computational device), or may include a plurality of computer processing devices. In some instances all processing will be performed by processing unit 1202, however in other instances processing may also be performed by remote processing devices accessible and useable (either in a shared or dedicated manner) by the system 1200.

Through a communications bus 1204 the processing unit 1202 is in data communication with a one or more machine readable storage (memory) devices which store instructions and/or data for controlling operation of the processing system 1200. In this example system 1200 includes a system memory 1206 (e.g. a BIOS), volatile memory 1208 (e.g. random access memory such as one or more DRAM modules), and non-volatile memory 1210 (e.g. one or more hard disk or solid state drives).

System 1200 also includes one or more interfaces, indicated generally by 1212, via which system 1200 interfaces with various devices and/or networks. Generally speaking, other devices may be integral with system 1200, or may be separate. Where a device is separate from system 1200, connection between the device and system 1200 may be via wired or wireless hardware and communication protocols, and may be a direct or an indirect (e.g. networked) connection.

Wired connection with other devices/networks may be by any appropriate standard or proprietary hardware and connectivity protocols. For example, system 1200 may be configured for wired connection with other devices/communications networks by one or more of: USB; FireWire; eSATA; Thunderbolt; Ethernet; OS/2; Parallel; Serial; HDMI; DVI; VGA; SCSI. Other wired connections are possible.

Wireless connection with other devices/networks may similarly be by any appropriate standard or proprietary hardware and communications protocols. For example, system 1200 may be configured for wireless connection with other devices/communications networks using one or more of: infrared; Bluetooth; Wi-Fi; near field communications (NFC); Global System for Mobile Communications (GSM), Enhanced Data GSM Environment (EDGE), long term evolution (LTE), wideband code division multiple access (W-CDMA), code division multiple access (CDMA). Other wireless connections are possible.

Generally speaking, and depending on the particular system in question, devices to which system 1200 connects—whether by wired or wireless means—include one or more input devices to allow data to be input into/received by system 1200 for processing by the processing unit 1202, and one or more output device to allow data to be output by system 1200. Example devices are described below, however it will be appreciated that not all computer processing systems will include all mentioned devices, and that additional and alternative devices to those mentioned may well be used.

For example, system 1200 may include or connect to one or more input devices by which information/data is input into (received by) system 1200. Such input devices may include keyboards, mice, trackpads, microphones, accelerometers, proximity sensors, GPS devices and the like. System 1200 may also include or connect to one or more output devices controlled by system 1200 to output information. Such output devices may include devices such as a CRT displays, LCD displays, LED displays, plasma displays, touch screen displays, speakers, vibration modules, LEDs/other lights, and such like. System 1200 may also include or connect to devices which may act as both input and output devices, for example memory devices (hard drives, solid state drives, disk drives, compact flash cards, SD cards and the like) which system 1200 can read data from and/or write data to, and touch screen displays which can both display (output) data and receive touch signals (input).

System 1200 may also connect to one or more communications networks (e.g. the Internet, a local area network, a wide area network, a personal hotspot etc.) to communicate data to and receive data from networked devices, which may themselves be other computer processing systems.

System 1200 may be any suitable computer processing system such as, by way of non-limiting example, a server computer system, a desktop computer, a laptop computer, a netbook computer, a tablet computing device, a mobile/smart phone, a personal digital assistant, a personal media player, a set-top box, a games console.

Typically, system 1200 will include at least user input and output devices 1214 and a communications interface 1216 for communication with a network such as network 106 of environment 100.

System 1200 stores or has access to computer applications (also referred to as software or programs)—i.e. computer readable instructions and data which, when executed by the processing unit 1202, configure system 1200 to receive, process, and output data. Instructions and data can be stored on non-transient machine readable medium accessible to system 1200. For example, instructions and data may be stored on non-transient memory 1210. Instructions and data may be transmitted to/received by system 1200 via a data signal in a transmission channel enabled (for example) by a wired or wireless network connection.

Applications accessible to system 1200 will typically include an operating system application such as Microsoft Windows®, Apple OSX, Apple IOS, Android, Unix, or Linux.

System 1200 also stores or has access to applications which, when executed by the processing unit 1202, configure system 1200 to perform various computer-implemented processing operations described herein.

In some cases part or all of a given computer-implemented method will be performed by system 1200 itself, while in other cases processing may be performed by other devices in data communication with system 1200.

Any flowcharts illustrated in the figures and described above define operations in particular orders to explain various features. In some cases the operations described and illustrated may be able to be performed in a different order to that shown/described, one or more operations may be combined into a single operation, a single operation may be divided into multiple separate operations, and/or the function(s) achieved by one or more of the described/illustrated operations may be achieved by one or more alternative operations. Still further, the functionality/processing of a given flowchart operation could potentially be performed by different systems or applications

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A system for delivering CBT-I to a patient, comprising:
one or more wearable devices each having one or more sensors configured to continuously monitor the patient's physiological, behavioural and/or environmental data;
a patient app executable on a patient system, the patient app including one or more CBT-I modules and being configured to receive physiological, behavioural and/or environmental data from the one or more wearable devices;
a clinician application executable on clinician system, the clinician application being configured to receive one or more CBT-I parameters pertinent to the CBT-I; and
a CBT-I server communicatively coupled to the patient app and to the clinician application, the CBT-I server being configured to make physiological, behavioural and/or environmental data received from the patient app available to the clinician application and selectively deliver the one or more of CBT-I modules on the patient app in accordance with one or more CBT-I parameters received from the clinician application.

2. The system according to claim 1, wherein the CBT-I server includes an analytics module configured to process physiological, behavioural and/or environmental data received from the patient app and CBT-I parameters received from the clinician application and generate a CBT-I regime that selectively delivers one or more of the CBT-I modules on the patient app.

3. The system according to claim 1, wherein the one or more wearable devices each include one or more sensory actuators, one of more of which are activated by one or more of the CBT-I modules during their delivery.

4. The system according to claim 3, wherein the one of more wearable devices and/or the patient app includes an acknowledgement module through which the patient acknowledges an activation of one or more of the one or more sensory actuators.

5. The system according to claim 3, wherein an activation of one or more sensory activators triggers a prompt to the patient on the patient app to enter a cognitive input into the patient app relevant to an event to which the activation of the one or more sensory activators relates.

6. The system according to claim 5, wherein activation of the one or more sensory activators is to awaken the patient and wherein the cognitive input relates to a quality and/or quantity of sleep that the patient experienced before being awakened.

7. The system according to claim 5, wherein activation of the one or more sensory activators is to deliver a sleep-restriction prompt to the patient and wherein the cognitive input relates to whether the patient is awake.

8. The system according to claim 1, wherein the one or more sensors are configured to detect the patient experiencing a hyperarousal event by monitoring changes in physiological data relative to a baseline, the patient app being configured, upon the detection of the hyperarousal event, to deliver a CBT-module to the patient that is selected to respond to the hyperarousal event.

9. The system according to claim 1, wherein the one or more sensors are configured to measure a quantity of light exposure to the patient, the patient app being configured to communicate light exposure measurements to the CBT-I server for on-communication to the clinician application.

10. The system according to claim 9, wherein the clinician application is configured to process the light exposure measurements by comparing the light exposure measurements to a light exposure goal previously stored at the clinician application or CBT-I server.

11. The system according to claim 2, wherein the patient app includes a diary module configured to receive from the patient, sleep and/or insomnia-related data points, the patient app being configured to communicate the sleep and/or insomnia-related data points to the analytics module for processing to generate the CBT-I regime that selectively delivers the one or more of the CBT-I modules on the patient app.

12. The system according to claim 1, wherein the CBT-I modules include modules for sleep application, sleep restriction, stimulus control, cognitive restructuring, light exposure management, relaxation techniques, goal setting, sleep coaching, sleep schedules and gamified components for delivering gamified cognitive brain training exercises.

13. The system according to claim 1, wherein the one or more sensors include a movement sensor, a respiration sensor for measuring one or more of respiratory rate, respiratory flow, respiratory volume, inspiration and expiration, a temperature sensor, a heart rate sensor, an SPO2 sensor, a galvanic skin response sensor, a light exposure sensor, a sweat sensor and a light sensor.

14. The system according to claim 13, wherein the patient app includes programming to receive sweat data from the sweat sensor and detect and quantify a level of hormone or caffeine present in sweat to which the sweat data pertains.

15. The system according to claim 1, wherein the one or more CBT-I parameters pertinent to the CBT-I received at the clinician application include sleep ability, prescribed time in bed, prescribed time to bed, prescribed time to wake, sleep efficiency and stimulus control methods.

16. A method for delivering CBT-I to a patient, comprising:
providing one or more wearable devices to the patient, each having one or more sensors configured to continuously monitor the patient's physiological, behavioural and/or environmental data;
executing a patient app on a patient system, the patient app including one or more CBT-I modules and being configured to receive physiological, behavioural and/or environmental data from the one or more wearable devices;
executing a clinician application on a clinician system, the clinician application being configured to receive one or more CBT-I parameters pertinent to the CBT-I; and
communicatively coupling a CBT-I server to the patient app and to the clinician application, the CBT-I server being configured to make physiological, behavioural and/or environmental data received from the patient app available to the clinician application and selectively deliver the one or more of CBT-I modules on the patient app in accordance with one or more CBT-I parameters received from the clinician application.

17. The method according to claim 16, wherein the CBT-I server includes an analytics module configured to process physiological, behavioural and/or environmental data received from the patient app and CBT-I parameters received from the clinician application and generate a CBT-I regime that selectively delivers one or more of the CBT-I modules on the patient app.

18. The method according to claim 16, wherein the one or more wearable devices each include one or more sensory actuators, one of more of which are activated by the one or more of the CBT-I modules during their delivery.

19. The method according to claim 18, wherein the one of more wearable devices and/or the patient app includes an acknowledgement module through which the patient acknowledges an activation of one or more sensory actuators.

20. The method according to claim 18, wherein an activation of one or more sensory activators triggers a prompt to the patient on the patient app to enter a cognitive input into the patient app relevant to an event to which the activation of the one or more sensory activators relates.

21. The method according to claim 20, wherein activation of the one or more sensory activators is to awaken the patient and wherein the cognitive input relates to a quality and/or quantity of sleep that the patient experienced before being awakened.

22. The method according to claim 21, wherein activation of the one or more sensory activators is to deliver a sleep-restriction prompt to the patient and wherein the cognitive input relates to whether the patient is awake.

23. The method according to claim 16, wherein the one or more sensors are configured to detect the patient experiencing a hyperarousal event by monitoring changes in physiological data relative to a baseline, the patient app being configured, upon the detection of the hyperarousal event, to deliver a CBT-module to the patient that is selected to respond to the hyperarousal event.

24. The method according to claim 16, wherein the one or more sensors are configured to measure a quantity of light exposure to the patient, the patient app being configured to communicate light exposure measurements to the CBT-I server for on-communication to the clinician application.

25. The method according to claim 24, wherein the clinician application is configured to process the light exposure measurements by comparing the light exposure measurements to a light exposure goal previously stored at the clinician application or CBT-I server.

26. The method according to claim 17, wherein the patient app includes a diary module configured to receive from the patient, sleep and/or insomnia-related data points, the patient app being configured to communicate the sleep and/or insomnia-related data points to the analytics module for processing to generate the CBT-I regime that selectively delivers the one or more of the CBT-I modules on the patient app.

27. The method according to claim 16, wherein the CBT-I modules include modules for sleep application, sleep restriction, stimulus control, cognitive restructuring, light exposure management, relaxation techniques, goal setting, sleep coaching, sleep schedules and gamified components for delivering gamified cognitive brain training exercises.

28. The method according to claim 16, wherein the one or more sensors include a movement sensor, a respiration sensor for measuring one or more of respiratory rate, respiratory flow, respiratory volume, inspiration and expiration, a temperature sensor, a heart rate sensor, an SPO2 sensor, a galvanic skin response sensor, a light exposure sensor, a sweat sensor and a light sensor.

29. The method according to claim 28, wherein the patient app includes programming to receive sweat data from the sweat sensor and detect and quantify a level of hormone or caffeine present in sweat to which the sweat data pertains.

30. The method according to claim 16, wherein the one or more CBT-I parameters pertinent to the CBT-I received at the clinician application include sleep ability, prescribed time in bed, prescribed time to bed, prescribed time to wake, sleep efficiency and stimulus control methods.
